(19) United States
(12) Patent Application Publication
(10) Pub. No.: US 2022/0023225 A1
(43) Pub. Date: Jan. 27, 2022

(54) SOFT SHELL GELATIN CAPSULES INCORPORATING IMMEDIATE AND EXTENDED RELEASE DOSAGE FORMS AND METHODS OF MANUFACTURING

(71) Applicants: Carlos Salazar Altamar, Barranquilla (CO); Gustavo Anaya, Soledad (CO); Braulio Teran, Barranquilla (CO); Newman Aguas Navarro, Barranquilla (CO); Wilmer Herrera, Barranquilla (CO); Pompilio Rafael Celedon Arrieta, Barranquilla (CO); Claudia Andrea Silva Blanco, Barranquilla (CO); Diego Rafael Monterroza Hernández, Barranquilla (CO); Rahumir Alfredo Gutierrez Castro, Barranquilla (CO)

(72) Inventors: Carlos Salazar Altamar, Barranquilla (CO); Gustavo Anaya, Soledad (CO); Braulio Teran, Barranquilla (CO); Newman Aguas Navarro, Barranquilla (CO); Wilmer Herrera, Barranquilla (CO); Pompilio Rafael Celedon Arrieta, Barranquilla (CO); Claudia Andrea Silva Blanco, Barranquilla (CO); Diego Rafael Monterroza Hernández, Barranquilla (CO); Rahumir Alfredo Gutierrez Castro, Barranquilla (CO)

(21) Appl. No.: 17/483,365

(22) Filed: Sep. 23, 2021

Related U.S. Application Data
(63) Continuation-in-part of application No. 17/384,670, filed on Jul. 23, 2021.
(60) Provisional application No. 63/055,435, filed on Jul. 23, 2020.

(51) Int. Cl. A61K 9/48; A61K 31/192; A61K 31/196; A61K 31/415

(52) U.S. Cl. CPC A61K 9/4825; A61K 9/4858; A61K 9/4866; A61K 31/415; A61K 31/192; A61K 31/196; A61K 9/4808

(57) ABSTRACT

A softgel capsule having incorporated within said capsule: (i) an active pharmaceutical ingredient in a liquid carrier exhibiting release profiles selected from the group consisting of immediate release, extended release and combinations thereof; and (ii) one or more smaller capsules or smaller solid dosage forms wherein said smaller capsules or smaller solid dosage forms have active ingredients that are compatible or not compatible with each other or with another active within the softgel capsule and wherein said smaller capsules or smaller solid dosage forms exhibit release profiles selected from the group consisting of extended release, and immediate release and combinations thereof.

This application is a continuation-in-part of U.S. application Ser. No. 17/384,670 filed Jul. 23, 2021; which application is incorporated by reference in it's entirety. This application also claims the priority benefit under 35 U.S.C. section 119 of U.S. Provisional Patent Application No. 63/055,435 entitled “Soft Shell Gelatin Capsules Incorporating Immediate And Extended Release Dosage Forms And Methods Of Manufacturing” filed Jul. 23, 2020; which is in its entirety herein incorporated by reference.

### FIELD OF THE INVENTION

This invention relates to softgels (or soft gelatin capsules) containing one or more smaller capsules or smaller solid dosage forms of active pharmaceutical ingredients within such capsules and to a process and apparatus for the manufacture thereof. The present invention also relates to a gelatin capsule of the soft type containing multiple active ingredients or the like, and more particularly to a novel gelatin capsule capable of containing multiple medicines or dietary supplement in the form of immediate and sustained release dosage forms as the content separated from each other, and its manufacturing method and manufacturing apparatus.

The present invention also relates generally to a method and apparatus for forming smaller capsules or smaller solid dosage forms having immediate and sustained release within capsules containing a measured amount of compatible and not compatible medicinals and more particularly to a method and apparatus for forming such capsules. The method and apparatus of the present invention are particularly useful in connection with forming softgel capsules having other smaller solid dosage forms containing multiple pharmaceutical products, such as for example medicines, vitamins, food supplements and the like which are compatible or not compatible with each other wherein said smaller dosage forms exhibit immediate or sustained release.

The present invention further relates to encapsulation machines and, more particularly, to soft encapsulation machines which make soft gelatin capsules having other smaller capsules within or other solid dosage forms which exhibit immediate or sustained release of the active pharmaceutical ingredient.

The invention is particularly useful for making formulations wherein two active ingredients are compatible or not compatible with each other but it is desirable to administer them in the same dosage form i.e., a capsule within a capsule or another solid dosage form within a capsule that exhibit optimized release properties.

### BACKGROUND OF THE INVENTION AND DESCRIPTION OF THE PRIOR ART

The art of encapsulation has been known for many years, particularly for the production of unit dosage forms containing various pharmaceutical products. Normally, such pharmaceutical capsules are composed of gelatin or some modification thereof, which is fabricated essentially into two different forms, namely, the so-called hard gelatin capsule and the soft gelatin capsule.

It is also known that conventional soft gelatin capsules are a preferred from of administration for medicaments and similar products; especially liquids, pastes, solids dispersed in liquids, or dry solids. Soft gelatin capsules also possess particular advantages for substances which require total protection from air and light, because the gelatin is completely sealed around the contents. An important example is for the encapsulation of vitamins, which has resulted in a high degree of stability thereof.

Hard gelatin capsules are also known in the art, and are generally formed from two distinct parts, namely the “cap” and the “body”, fitting one into the other so as to form the complete capsule. The cap and the body are manufactured by the same process consisting of immersing in a gelatin solution the end of a mandrel whose form corresponds to the inner volume of the cap or of the body, then withdrawing the mandrel from the solution and letting the layer of gelatin thus deposited dry, which is then removed like a glove finger. Hard shell capsules so formed have problems of leakage and do not provide adequate protection from air and light

Soft gelatin capsules, now more commonly known as softgels, have been well known and widely used for many years. Softgels generally comprise an outer shell primarily made of gelatin, a plasticizer, and water, and a fill contained within the shell. The fill may be selected from any of a wide variety of substances that are compatible with the gelatin shell. Softgels are widely used in the pharmaceutical industry as an oral dosage form containing many different types of pharmaceutical and vitamin products. In addition to use as an oral dosage form for drugs and vitamins, soft gelatin capsules or softgels are also designed for use as suppositories for rectal or vaginal use. Other uses are for topical and ophthalmic preparations and the like. The cosmetic industry also uses softgels as a specialized package for various types of perfumes, oils, shampoos, skin creams and the like. Softgels are available in a great variety of sizes and shapes, including round shapes, oval shapes, oblong shapes, tube shapes and other special types of shapes such as stars. The finished capsules or softgels can be made in a variety of colors. Also, opacifiers may be added to the shell.

The process for making softgel capsules includes the step wherein the gelatin shell and the fill material come together to form Softgel capsules. It takes place in a closed environment called clean room where the relative humidity is around 20%. The gelatin shell and fill material are brought together simultaneously in the encapsulation machine.

The process is basically performed as follows: a pump delivers the warm gelatin over two chilled drums which are located at both opposite sides of the machine, through a spreader box that sits over each drum. The warm liquid gelatin flows over the drums and this transforms the liquid gelatin into two solid ribbons of gel. The left and right ribbons pass over rollers which feed them through two die rolls. These die rolls determine the shape and size of softgels and cut the Softgel shell from the ribbons as they turn around.

Simultaneously, a sensitive and high accuracy positive displacement pump delivers the fill material into a heated wedge which sits between rotary dies. This wedge injects the fill material into the die cavities between ribbons just right before the die rolls cut the ribbons and seal the two halves together. Warm just formed softgels slide gently through a chute onto a conveyor belt which carries them to the tumble dryer where cooling and drying process takes place.

In more specific detail, typical soft encapsulation machines form at least two flexible gelatin sheets or ribbons by cooling molten gelatin on separate drums then lubricating and guiding the sheets into communication with each other over co-acting dies while simultaneously dispensing a desired quantity of fill material between the sheets in synch with cavities in the outer surfaces of the dies to produce soft capsules. The encapsulation machines typically utilize gearing to control the relative rotations of the various components and fill mechanisms to synchronize the operation of these various components. The synchronization of these various components, however, can vary depending upon a variety of factors, such as the particular dies used, the number of cavities and the size of the cavities on the dies, and the type of material used to form the sheets. To change the synchronization of the various components, mechanical gears are required to be changed to obtain the desired ratios and synchronization of these components. The changing of gears, however, is time intensive. Additionally, the use of mechanical gears provides finite gear ratios which limit the synchronization of the various components to the mechanical gears that are available. Thus, it would be advantageous to provide a capsule machine wherein the synchronization and rates at which the various components operate can be altered without the necessity of changing gears. Additionally, it would be advantageous if the synchronization between the various components can be infinite to thereby allow more precise synchronization between the various components. It would also be advantageous to allow various components, such as the fill mechanism, to be adjusted independently of the other components while the machine is running to allow for adjustments of the timing of fill material inserted into each of the soft capsules. It would also be advantageous to eliminate the use of casting drums in the making of softgel capsules.

During the operation of the capsule making machine, the contact between the adjacent dies can be adjusted by the operator of the capsule making machine. Typically, the operator is able to move one of the dies closer to the other die so that the pressure or force exerted on the sheets passing between the adjacent dies can be adjusted. Such adjustments, typically are mechanical adjustments made by fluid actuators, such as pneumatic cylinders. The operator is able to adjust the pneumatic pressure thereby altering the force the dies exert on one another and on the sheets. This adjustability allows an operator to customize the pressure to ensure that quality soft capsules are produced. However, the dies are susceptible to premature failure and/or wear when the pressure or force between the two dies is more than that required to produce acceptable soft capsules. Thus, it would be advantageous to monitor/record the pressure applied to the dies so that quality capsules are produced without inducing excessive wear or premature wear on the dies.

A material fill mechanism is used to supply the fill material that is encapsulated within the soft capsules. When the fill material is a liquid, such as a liquid medication or die for a paint ball capsule, the fill mechanism includes a plurality of positive displacement plunger-type pumps that are arranged in a housing above the dies. The plunger-type pumps are positioned on a yoke that moves linearly in a reciprocating motion to allow the plunger-type pump to fill with the liquid fill material on one stroke and subsequently discharge the liquid fill material on the other stroke. A valving arrangement between opposing pumps is utilized to control the discharge and filling of the pumps. The valve arrangement includes a sliding member that moves linearly back and forth in a direction generally perpendicular to the linear motion of the yoke. The discharge of the liquid fill material into the sheets as they are passing through the dies is coordinated with the operation of the dies to insure that the timing of the injection of the liquid fill material is synchronized with the cavities on the dies. Typically, this synchronization has been performed through the use of mechanical gears that link the timing of the stroke to the rotation of the dies. Thus, in order to adjust the synchronization a mechanical gear change is required which is time consuming. Additionally, the timing is limited to a finite number of gear ratios as determined by the gears that are available.

The sliding member of the valving mechanism requires lubrication. Typically, the lubrication is provided by a lubricating pump with its own separate drive. However, the use of a separate drive to operate the lubricating pump adds additional complexity and components to the capsule machine. Thus, it would be advantageous if a motion of the slide member and/or the yoke could be utilized to drive the lubrication pump.

The pumps are typically contained within a housing that is filled with a lubricating oil that is used to lubricate the sliding member. The pumps, however, can leak around their seals and contaminate the lubricating oil with the leaking fill material. Contamination of the oil requires a time consuming and possibly difficult clean up and can cause the lubricating oil to not perform as designed thereby increasing the wear on the sliding surfaces and decreasing the life span of the sliding surfaces. Thus, it would be advantageous to capture any fill material that leaks from the pumps and deter or prevent the liquid fill material from contaminating the lubricating oil within the pump housing.

The pumps are typically driven by a drive mechanism that is also located within the pump housing. Because the drive mechanism is located in the pump housing, it is possible for liquid fill material that leaks from the pumps to contaminate not only the lubrication oil but also the drive mechanism. When switching from one fill material to another, the pump and all of the components in the pump housing are required to be thoroughly cleaned to remove all contamination. The locating of the drive mechanism within the pump housing provides additional components that must also be cleaned when changing the fill material. Thus, it would be advantageous to separate the drive mechanism from the pump housing to reduce the components that are required to be cleaned when changing fill material.

The soft capsules produced by the encapsulation machine are transported from the encapsulation machine to a dryer to additionally dry the soft capsules and to make them into final form.

Subsequent to the rotary die process used to produce the gelatin shells having a medicament fill therein, the resulting capsules are typically washed with a solvent that evaporates easily. Thereafter, the capsules are typically tumble dried in a series of hollow drums with perforated walls. Heated dry air is continuously pumped through the rotating drums at an air temperature typically less than 35° C. The warm air blown into the capsules appears to penetrate the shell and cause it to dry from the inside by moving the water outward to the surface of the capsule. By the time the capsules exit this process, all of the solvent used in washing has typically been evaporated, and a large proportion (50-60%) of the water from the gelatin shell has been removed. Recent developments in drying include bypassing the drum drying stage and having the capsules dried in a drying tunnel or room as further discussed below.

After the capsules exit the last drying drum, the capsules are typically spread on drying trays. The final drying phase for softgels is typically accomplished by passing the drying trays through drying tunnels or into drying rooms. Stacks of trays are inserted into drying tunnels or drying rooms, in which controlled temperature air (21°-24° C.) and low relative humidity (20-30%) is continuously circulated. Although additional water may be removed from dry capsules by further heating, for example at 40° C., such a procedure has not been found to be practical or necessary. See Van Hostetler and J. Q. Bellard in The Theory and Practice of Industrial Pharmacy, “Capsules”, (1970), Chapter 13 at pages 346-383, and in particular at page 380.

The drying time, for most softgels, is 16-24 hours, but may be slightly longer if the softgels are over 20 minims in size or if the softgels contain a non-oily type liquid base. Softgels permitted to come to water equilibrium in this controlled environment are considered “dry”. The gelatin fill and shell of such “dry” softgels contain 6-10% water depending on the specific gelatin and fill formula used. After drying, the capsules are typically inspected and finished using varied known techniques.

Applicant is aware of the following publications briefly discussed below. U.S. Pat. No. 1,970,396 features a method and machine for producing soft gelatin capsules in an automated process. The method involves the formation of two gelatin sheets or films through the use of a gravity fed spreader box, cooling the liquid gelatin on two separate webs, then lubricating and guiding the two sheets into communication with each other between two co-acting dies while simultaneously dispensing the proper amount of medicine or other filling material between the sheets in registration with half cavities in the outer surface of the dies.

U.S. Pat. No. 5,761,886 discloses an apparatus for forming capsules that provides rotary dies that are independently moveable and the ability to vary the speed of the dies during the formation of a single capsule. The '886 device also utilizes independently controlled casting drums to reduce “set-up” time and provide better quality control. Even though the '886 patent discloses a very sophisticated encapsulation machine, it still utilizes a gravity fed spreader box for formation of the encapsulating ribbon.

Other patents relating to encapsulation techniques which disclose the use of spreader boxes to create the film or ribbon on a casting drum include U.S. Pat. Nos. 2,288,327; 2,774,988; 5,246,638; 5,735,105; and 6,022,499.

Many shell and fill formulations are discussed in Van Hostetler and J. Q. Bellard noted below as well as in “Advances in Softgel Formulation Technology”, M. S. Patel, F. S. S. Morton and H. Seager, Manufacturing Chemists, July 1989; “Soft Elastic Gelatin Capsules: A Unique Dosage Form”, William R. Ebert, Pharmaceutical Technology, October 1977; “Soft gelatin capsules: a solution to many tableting problems”, H. Seager, Pharmaceutical Technology, September 1985; U.S. Pat. No. 4,067,960 to Fadda; U.S. Pat. No. 4,198,391 to Grainger; U.S. Pat. No. 4,744,988 to Brox; and U.S. Pat. No. 4,780,316 to Brox. All of the above references are incorporated herein by reference.

Soft gelatin capsules serve chiefly for the containment of liquids, i.e. oily solutions, suspensions or emulsions. Vegetable, animal and mineral oils, liquid hydrocarbons, ethereal oils and also polyethylene glycols are in use as fillings. Fats and waxes are also applied or admixed to increase the consistency. Polyethylene glycols are superior to other possible filling materials for soft gelatin capsules in a number of ways. In contrast to oily liquids, polyethylene glycols are mixable with water in all proportions.

At the same time, because polyethylene glycols are able to dissolve many drugs which are sparingly soluble or insoluble in water, the use of polyethylene glycols with such drugs makes possible a particularly favourable liberation of the active material. In many cases, sparingly water soluble drugs which have been dissolved in polyethylene glycols and then put into soft gelatin capsules are outstanding, by virtue of an exceptionally good bio-availability of the drug.

The present invention fulfills a long felt need of providing softgel capsules incorporating within the capsule smaller dosage forms that exhibit different release properties depending on the desired therapeutic effect.

### OBJECTS OF THE INVENTION

It is an object of the present invention to provide a new anti-inflammatory compounds.

It is also an object of the present invention to provide a new solvent system useful for filling softgel capsules.

It is a specific object of the present invention to provide solutions containing non-steroidal anti-inflammatories dissolved in glycofurol.

Other objects and embodiments of the present invention will be discussed below. However, it is important to note that many additional embodiments of the present invention not described in this specification may nevertheless fall within the spirit and scope of the present invention and/or the claims.

### BRIEF DESCRIPTION OF THE FIGURES

FIG. 1 illustrates the extended dissolution profiles of the Tablet cores using a hydrophilic polymer at different viscosities of the polymer for obtaining an extended release matrix.

FIG. 2 shows as example the dissolution profile of Unigel biphasic release from an immediate release fill content and an extended release coated Tablet releasing the API up to 10 hours.

FIG. 3 describes the effect of the thickener concentration on the % of dissolved API BCS class II as function of the thickener content.

FIG. 4 features the dissolution profiles of the coated extended release Tablets of two core formulations F1 and F2 up to 10 hours.

FIG. 5 illustrates the extended release dissolution profiles of Sodium Diclofenac 100 mg soft gel capsule (SGC) with ratio coating 1:coating 2 with a weight gain 5%.

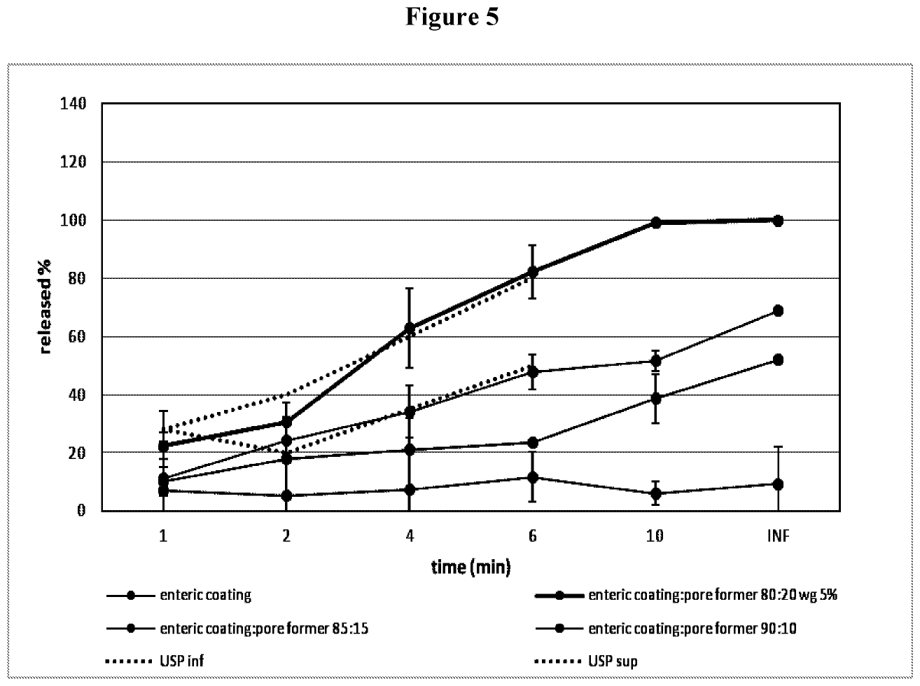

FIG. 6 describes the extended release dissolution profiles of Sodium Diclofenac 100 mg soft gel capsule (SGC) with ratio coating 1:coating 2 80:20 and weight gain 5-15%.

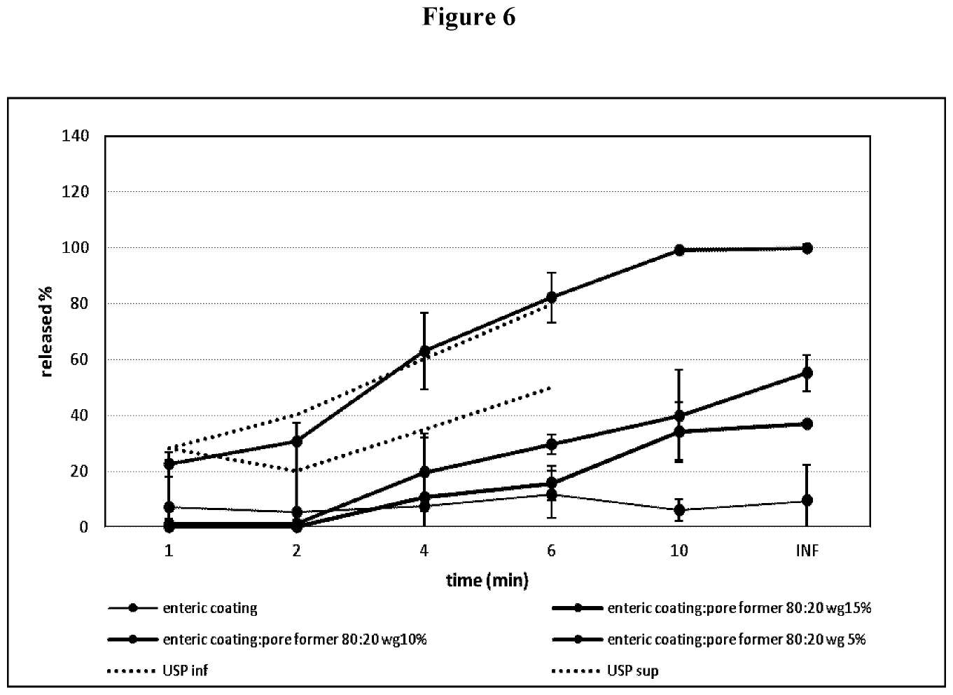

FIG. 7 shows the dissolution profiles of Sodium Diclofenac 100 mg soft gel capsule having to solid dosage forms (25 mg IR+75 mg ER Tablet).

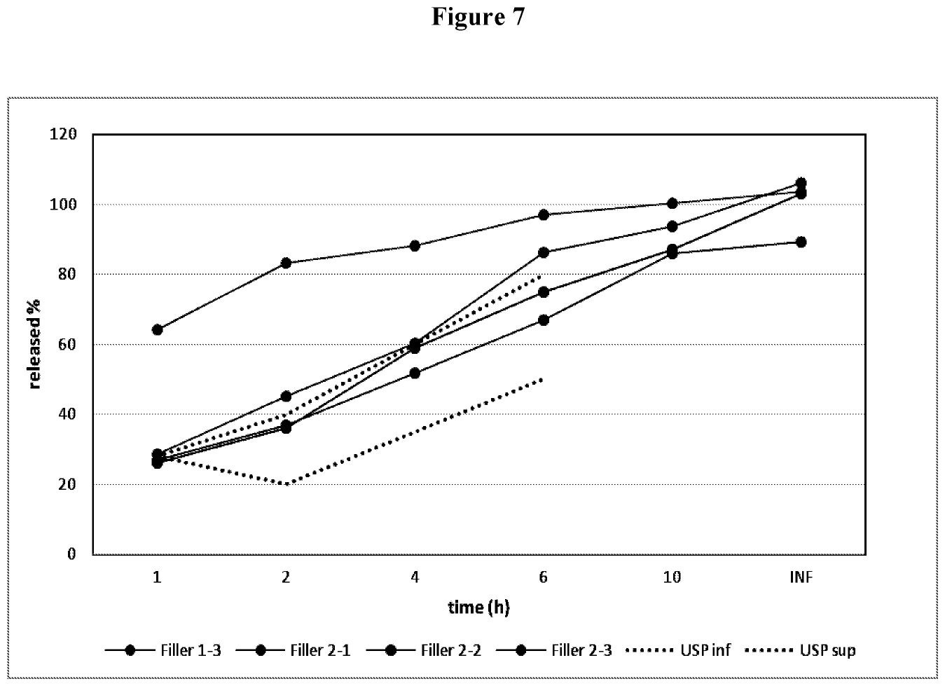

FIG. 8 illustrates the dissolution extended profiles for several diclofenac formulations having starch and lactose as matrix material for making tablets.

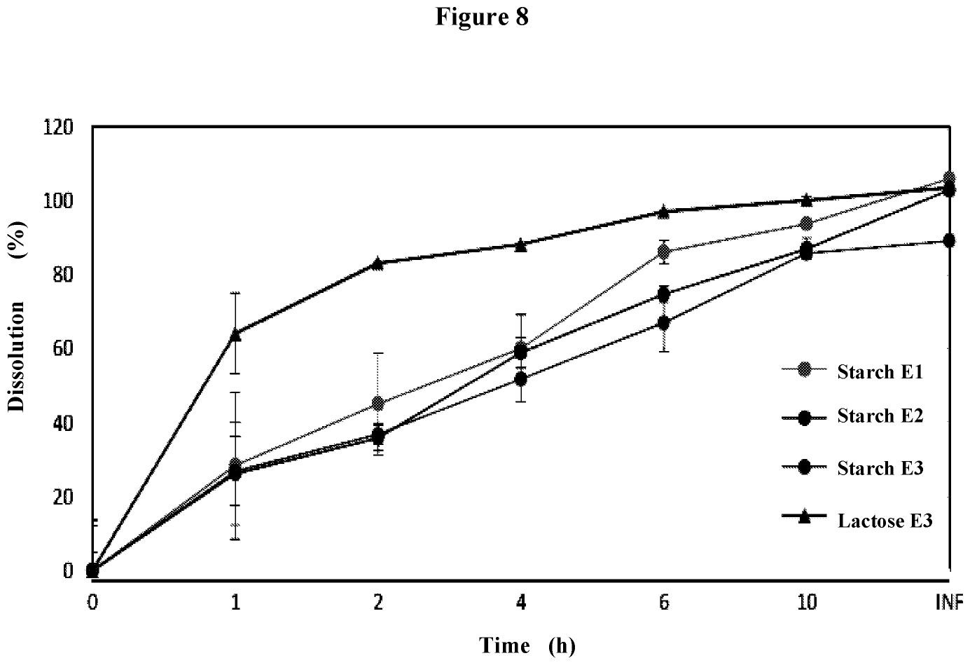

FIG. 9 is a front view of the complete apparatus of the invention showing all the elements of the apparatus.

FIG. 10 is also a front view of the apparatus of FIG. 1 without the spreader boxes and casting drums.

FIG. 11 is a front view of the mechanism for filling the capsules with other capsules.

FIG. 12 is also a front view of how the smaller capsules are dispensed into the larger capsule.

FIG. 13 shows the smaller capsule hopper having capsules which are fed via guiding channels into the larger capsule.

FIG. 14 shows a representative end product of the invention containing two capsules inside another capsule.

FIG. 15 are representative examples of products contemplated by the invention.

FIG. 16 illustrates products containing a single ER tablet or ER bead.

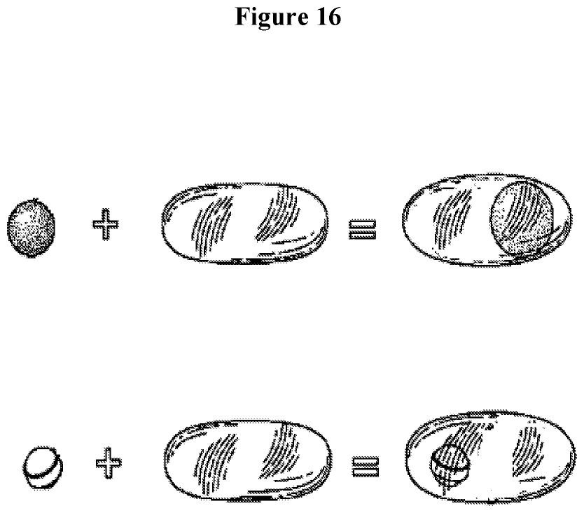

### SUMMARY OF THE INVENTION

The present invention provides softgel capsules having incorporated within said capsule: (i) an active pharmaceutical ingredient in a liquid carrier exhibiting release profiles selected from the group consisting of immediate release, extended release and combinations thereof; and (ii) one or more smaller capsules or smaller solid dosage forms wherein said smaller capsules or smaller solid dosage forms have active ingredients that are compatible or not compatible with each other or with another active within the softgel capsule and wherein said smaller capsules or smaller solid dosage forms exhibit release profiles selected from the group consisting of extended release, and immediate release and combinations thereof.

The invention also provides a softgel capsule having incorporated within said capsule: (i)

an immediate release active pharmaceutical ingredient in a liquid carrier; and (ii) one or more smaller capsules or smaller solid dosage forms wherein said smaller capsules or smaller solid dosage forms have active ingredients that are compatible or not compatible with each other or with another active within the softgel capsule and wherein said smaller capsules or smaller solid dosage forms exhibit release profiles selected from the group consisting of extended release, and immediate release and combinations thereof.

The present invention responds specifically to the long-felt need heretofore unmet by the prior art, and especially with a view to overcoming the inherent inadequacies of combination of pharmaceuticals that are compatible or not compatible for oral delivery to mammals. The composition is a pharmaceutical combination i.e., a capsule or a solid form within a capsule providing the convenience and reliability of oral administration, while providing near simultaneous delivery in vivo of compatible and incompatible substances which also exhibit immediate and extended release profiles. The composition is shelf stable when formulated.

The foregoing, and other advantages of the present invention, are realized in one aspect thereof in an oral pharmaceutical composition that is a combination of compatible and incompatible active ingredients. The composition comprises a soft capsule which includes one pharmaceutical in a first capsule or solid form having extended release profile which is enclosed in a second soft capsule also containing a second active ingredient which exhibits an immediate release profile. The soft capsules are preferably made of gelatin. The active ingredients may be combined with acceptable grade carriers.

In another aspect, the invention is a method of simultaneously delivering compatible and incompatible compounds to mammals in vivo. Such delivery is achieved by administering orally to a mammal a soft capsule containing a first substance in a first capsule or in a solid form, which is enclosed with a second substance, incompatible with the first substance, in a second larger soft capsule. Like stated above the resulting dosage forms are designed to exhibit immediate release or extended release as desired.

In another embodiment, this invention provides a method for preparing shelf-stable compositions of incompatible substances, which includes the use of multiple capsules or solid dosage forms of variable composition. Such method is accomplished manually or by the apparatus of the invention further described below.

As used herein, the term “incompatible” is meant to refer to substances which deleteriously react with one another when combined in desired levels or concentrations.

The invention also provides an apparatus for making softgel capsules having incorporated therein other solid dosage forms selected from the group consisting of pellets, smaller capsules, smaller tablets, sustained release solid dosage forms, immediate release solid dosage forms, extended release solid dosage forms and zero order release solid dosage forms, said apparatus comprising: (a) two spreader boxes; (b) two casting drums; (c) a pair of rotary dies having means for suction; (d) a liquid fill system; (e) a wedge for heating gelatine ribbons and feeding said fill; and (f) two lateral dispensing devices said lateral dispensing devices including hoppers having said solid dosage forms, channelguides for transporting said solid dosage forms and a grasping claw for dispensing said solid dosage form into the softgel pocket formed in the rotary dies.

The invention further provides a dispensing device for dispensing and feeding solid dosage forms into a softgel capsule said dispensing and feeding device including a hopper having said solid dosage forms, channelguides for transporting said solid dosage forms and a grasping claw for dispensing said solid dosage form.

The instant invention also provides a method for making softgel capsules having incorporated therein other solid dosage forms, said method comprising: casting a gel forming composition to make films; (b) passing said films through a pair of rotary dies having vacuum means to make pockets; (c) feeding smaller solid dosage forms into said pockets using a lateral dispensing and feeding system that uses a grasping claw; (d) filling said pockets with a medicine formulation in liquid form via a wedge segment; and (e) forming said capsule by sealing the pockets together and wherein said smaller dosage forms exhibit immediate and extended release profiles.

The invention is also a process for making a softgel capsule having incorporated therein another capsule, said process comprising: (a) feeding film sheets between a first die roll and a second die roll wherein each of the die rolls have capsule pockets in a plurality of rows and said capsule pockets have at least one orifice for application of suction; (b) applying suction while said film is in place in the capsule pockets; (c) feeding via guidechannels through a lateral dispensing device having a hopper and a grasping claw preformed smaller capsules onto the film sheets overlying the die rolls at positions having the capsule pockets; (d) filling said capsule pockets also via a wedge segment with a liquid medical formulation; and (e) cutting the film sheets about the capsule pockets to form said soft gel capsules having capsules in combination with a suitable liquid pharmaceutical combination.

The invention further provides softgel capsules incorporated into an outer softgel capsule, tablets incorporated into an outer softgel capsule, microgranules incorporated into an outer softgel capsule, and any combination between softgels, tablets and/or microgranules incorporated into an outer softgel capsule.

The instant invention also provides a softgel capsule having incorporated therein another solid dosage form selected from the group consisting: (a) one capsule contains an omega oil and the other solid dosage form is a capsule having a statin; (b) one capsule contains a non-steroidal antiinflammatory and the other solid dosage form contains and antihistamine; and (c) one capsule contains and omega oil and the other solid dosage form contains a salicylate.

The smaller capsules or smaller solid dosage forms incorporate drugs having activity against all diseases known to human kind. The smaller solid dosage forms typically exhibit immediate release, delayed release, extended release, sustained release and combinations thereof.

Other advantages and a fuller appreciation of the specific adaptations, compositional variations, and physical and chemical attributes of the present invention will be gained upon an examination of the following detailed description of the invention, taken in conjunction with the accompanying drawings and appended claims.

### DETAILED DESCRIPTION OF THE INVENTION

The present invention provides softgel capsules having incorporated within said capsule: (i) an active pharmaceutical ingredient in a liquid carrier exhibiting release profiles selected from the group consisting of immediate release, extended release and combinations thereof; and (ii) one or more smaller capsules or smaller solid dosage forms wherein said smaller capsules or smaller solid dosage forms have active ingredients that are compatible or not compatible with each other or with another active within the softgel capsule and wherein said smaller capsules or smaller solid dosage forms exhibit release profiles selected from the group consisting of extended release, and immediate release and combinations thereof.

The invention also provides a softgel capsule having incorporated within said capsule: (i) an immediate release active pharmaceutical ingredient in a liquid carrier; and (ii) one or more smaller capsules or smaller solid dosage forms wherein said smaller capsules or smaller solid dosage forms have active ingredients that are compatible or not compatible with each other or with another active within the softgel capsule and wherein said smaller capsules or smaller solid dosage forms exhibit release profiles selected from the group consisting of extended release, and immediate release and combinations thereof.

The present invention provides an innovative and efficient system for the manufacture of capsules with two or more internal components. Although the internal components may be incompatible the invention is also intended to provide internal components that are compatible but are intended to be released at different intervals.

The present invention provides an advanced drug delivery system that places different pharmaceuticals forms in a single dosage combination. The invention allows delivering incompatible pharmaceutical actives in the form of solid, liquid, microgranules, gels, hard shell or soft gel capsules within an outer softgel capsule.

The novel dosage system allows for combining different therapeutic entities that have never been combined before, via oral, ovules, or suppositories.

For the multi-drugs regimen patients and due to the incompatibility of some actives that can not be combined in a single dose, the instant invention offers a universe of possibilities for current and future new drugs combinations and supplies different releasing delivery.

In the present invention, existing and proven delivery systems are combined in a highly reliable, easy to use and affordable manufacture that give the resulting dosage form unique characteristics to deliver single or multiple APIs regardless of physical-chemical compatibility and/or stability liabilities.

For the multi-drugs regimen patients this delivery system is a viable alternative; due to the manufacturing of IR plus MR combinations in tablets and hard-gelatin capsules while enhancing dosing accuracy and by-passing dissolution barriers and coating issues. It allows the formulation of combination products, highly needed to assure patient compliance and allow synergic clinical effects in a safe and stable dosage form.

Some of the most important advantages are:

Fast and sustained release in a single dose.
Gastric or intestinal release in the same dose.
Fewer intakes to be administered.
Simplicity of regimen reduces mistakes.
Impossible to be falsified.
Reduces number of Rx's prescribed by Physician.
Smaller number of presentations to maintain.

The invention further provides soft-gelatin capsules as a immediate-release (IR) delivery system, that upon rupture, it releases immediate or modified release (MR) tablets or extended release, capsules, softgels, granules and/or microgranules. Compatible and/or incompatible pharmaceutical active ingredients, and/or blends of IR and MR or ER dosage forms of the same or different active pharmaceutical ingredients (APIs) can be dosed simultaneously in a single capsule. These capsules may be designed to be administered orally, vaginally or rectally, as needed.

Polymeric compositions have been widely used as a matrix for extended or sustained drug release formulations. For such applications, a highly hydrophilic polymeric composition is suitably employed. Cellulose ethers such as methyl cellulose and hydroxypropyl methylcellulose are among the polymeric compositions which have been most widely used in this manner. Other cellulose ethers, such as ethyl cellulose, methyl cellulose, hydroxypropyl cellulose, hydroxyethyl cellulose, sodium carboxymethyl cellulose, have also been used. They exhibit fast hydration forming a protective initial gel layer quickly through which the drug is released to the system. Once the initial gel layer is formed, it continues to allow additional water to penetrate into the mass. Soluble materials will wet, dissolve, and diffuse out of the matrix while insoluble materials will be held in place until the surrounding complex erodes or dissolves away. As the outer gel layer begins to fully hydrate and be dissolved, a new layer replaces it that is tight and strong enough to retard diffusion and sustain uniform drug release. Factors affecting the rate of hydration of the polymeric composition, and thereby the drug release rate, include its viscosity, concentration, particle size, and chemical makeup.

Another factor affecting the rate of gel formation or hydration of the polymeric composition used as an extended drug release matrix is the chemical characteristics of the drug employed. Certain polymers can be employed effectively for some drugs but not for others. The degree of water solubility of the drug, molecular weight of the drug, and the diffusion coefficient of the drug in hydrated polymer are critical.

The oral dosage unit of the embodiments of the present invention also contain one or more compositions such as diluents or fillers which are therapeutically inert and pharmaceutically acceptable and provide bulk. Examples of such diluents or fillers include cornstarch, lactulose, dextrose and the like.

The extended release oral dosage unit can be in the form of a tablet or a capsule. Tablets may be prepared or manufactured in accordance with an embodiment of the present invention on any conventional tableting equipment.

In the preparation of the oral extended release formulation of the present invention, diclofenac tablets are ground into a fine powder and mixed with one or more cellulose ethers and one or more diluents or fillers and tableted and inserted into a soft gelatin capsule. The amount of diclofenac that is included per oral dosage unit may vary widely. The therapeutically effective dose range of about 0.025 mg to about 0.40 mg per unit is preferred to control most of the symptoms of the clinical disorders which diclofenac may benefit. The dose of the oral dosage unit can be exactly specified, however, as required.

The cellulose ethers or mixtures thereof employed as the extended release matrix in the present invention are ultra-fine, rapidly hydrating formulations having a number average molecular weight of at least 86,000 or a 2% aqueous solution of viscosity of at least 4000 cps and wherein at least 90% by weight of the cellulose ether particles can pass through a 100 mesh screen. An important aspect of the present invention is that the extended release profile of diclofenac can be specified by the types or amounts of cellulose ethers used. The invention is thus very adaptable and versatile to each particular use. The oral dosage formulation herein described provides a preferred release period suitable for the dosing of diclofenac twice per day, at twelve hour intervals.

A functionally effective amount of the cellulose ether composition is employed. Such an amount is an amount sufficient to extend the release of diclofenac for up to twelve hours. Such an amount can vary and typically ranges from about 30 to about 70 weight percent, and preferably from about 30 to about 40 weight percent based on the weight of the solid dosage form, although any functionally effective amount can be employed.

One preferred extended release matrix is hydroxypropyl methylcellulose such as Methocel®, which is manufactured by the Dow Chemical Company, U.S.A. The preferred Methocel® for an eight hour release period is E4M which has a hydroxypropoxyl substitution of from about 7 to about 12 weight percent, a methoxyl substitution of from about 28 to about 30 weight percent, a number average molecular weight of about 86,000, a 2% aqueous solution of viscosity of about 4000 cps and 95% by weight can pass through a 100 mesh screen. The preferred Methocel® for a twelve hour release period is K100M which has a hydroxypropoxyl substitution of from about 7 to about 12 weight percent, a methoxyl substitution of from about 19 to about 24 weight percent, a number average molecular weight of about 246,000, a 2% aqueous solution of viscosity of about 100,000 cps and at least 90% by weight can pass through a 100 mesh screen.

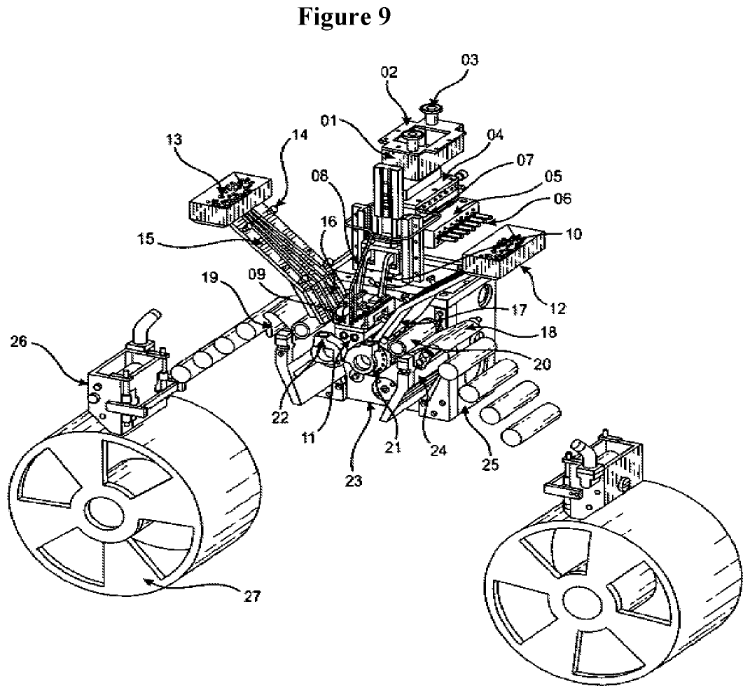

Referring in detail to the apparatus shown in FIG. 9, reference numeral 1 illustrates a medicine hopper having a cover 2 and a medicine feeder 3 connected with a clamp. The apparatus further includes a medicine distributor system 4, pump 5 to pump medicine and further includes plunger 6. The apparatus also includes a fitting distributor connection 7, medicine tubing/hoses 8, a segment coupling connection 9, a support segment 10, and wedge segment 11.

The apparatus has lateral hoppers 12 and 13 containing smaller capsules or other solid dosage forms that are intended to be encapsulated by the soft gels being formed in the rotary dies. The lateral hopper dispensing system includes acrylic or other material knob fasteners 14 and acrylic substrate 15 having guide channels/tracks 16 for the smaller capsules or other smaller solid dosage forms such pellets or minitablets, etc. The lateral dispensing system of the invention includes a grasping claw 17 for dispensing the smaller capsules coming through channels/track 16. The apparatus further includes the conventional aspects of making softgel capsules which includes a gelatin film 18, guiding rollers 19, tensioner 20, rotary mold 21, a vacuum system 22, capsule exit 23 after the capsule is formed, a yoke support arm 24, housing 25, spreader gel dispensing boxes and casting drum 27.

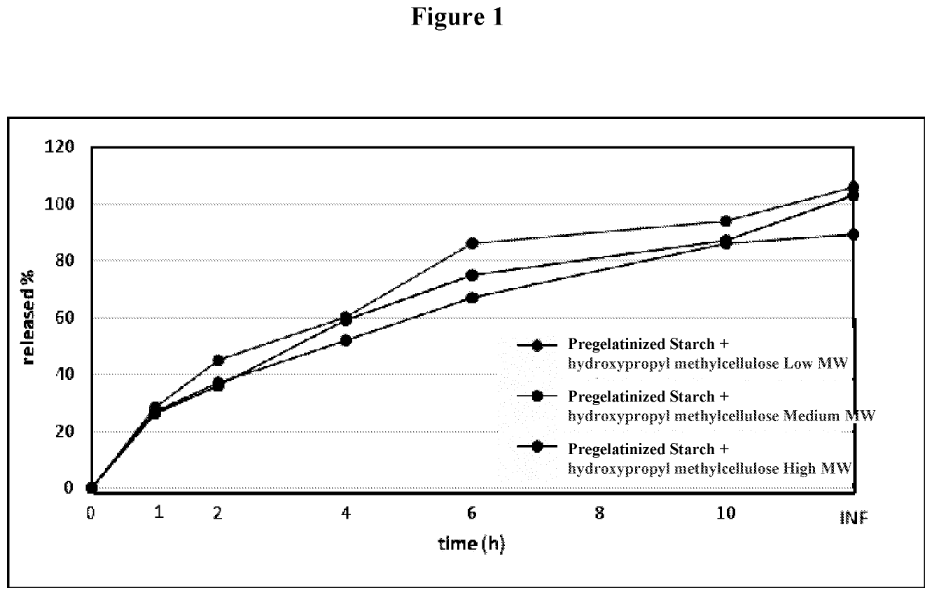

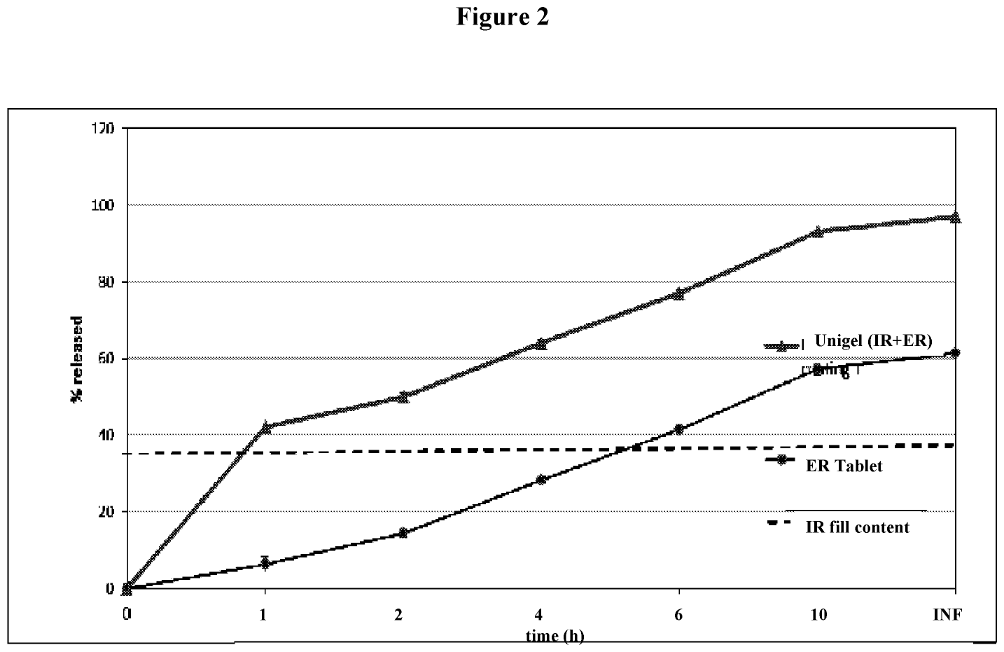

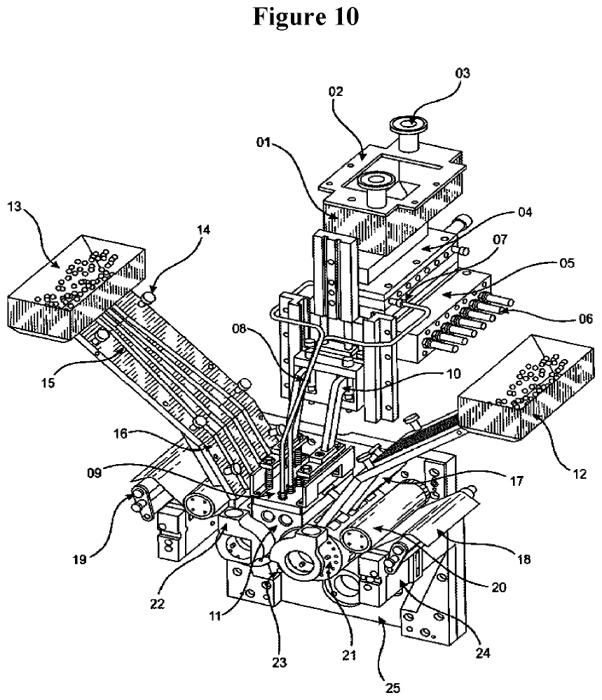

FIG. 10 illustrates the apparatus of FIG. 9 without the spreader gel dispensing boxes and casting drums. The reference numerals in FIG. 2 are identical as those in FIG. 1.

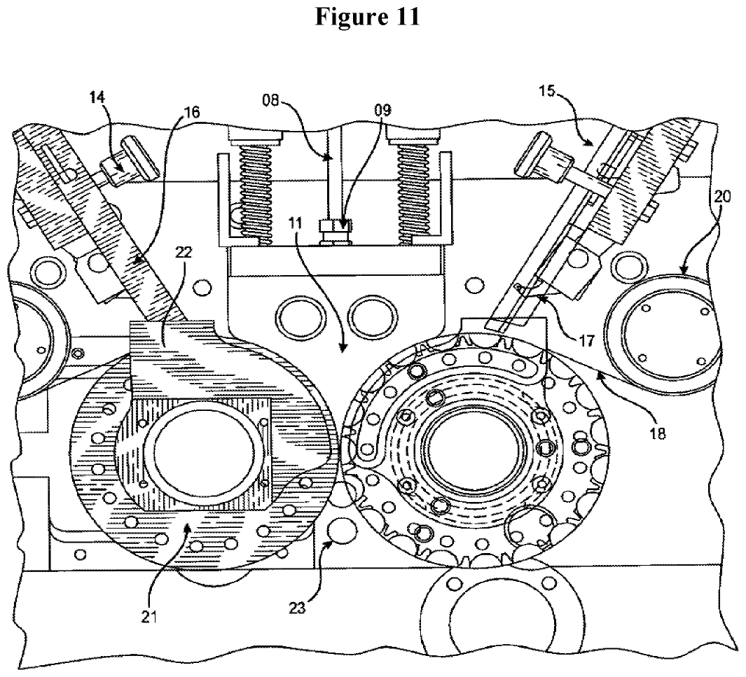

FIG. 11 shows the dispensing and feeding of solid dosage forms or capsules that come from hoppers 12 and 13 (not shown-See FIGS. 1 and 2) controlled by grasping claw 17 with volume capacity for accurate dosing fixed within the capsule. The smaller dosage form or smaller capsules is fed through guide channels 16 and deposited inside a half pocket as the softgel capsule is being formed in rotary die 21. The grasping claw 17 releases each capsule into each packet as the rotary die moves. The final capsule is also filled with additional pharmaceutical actives in liquid form injection tubing 8. After filling the formed capsule 23 falls-through to a conveyor belt and then transported for drying.

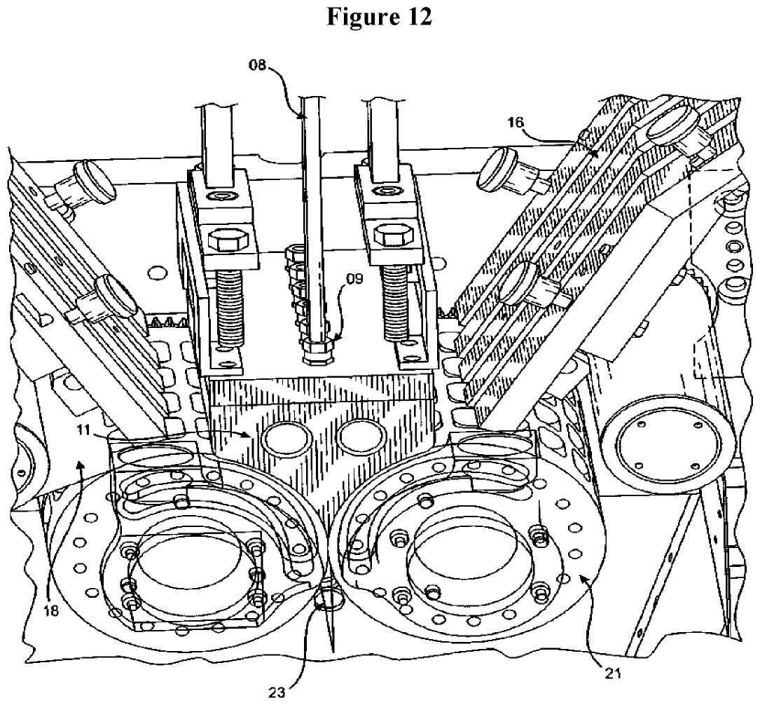

FIG. 12 further illustrates in more details the feeding of solid dosage forms or capsules into the rotary molding process for making softgel capsules containing internally other dosage forms such as smaller capsules, pellets, small tablets, etc. The feeding of the internal capsule is made by an independent dispenser having guide channels 16 so that as capsules are deposited in the pocket of the rotary die/mold 21, the wedge segment 11 is used to simultaneously dispense a liquid medicine product to fill the capsule. As is well known gelatin film 18 is used to form the softgel pocket in the rotary die/mold 21.

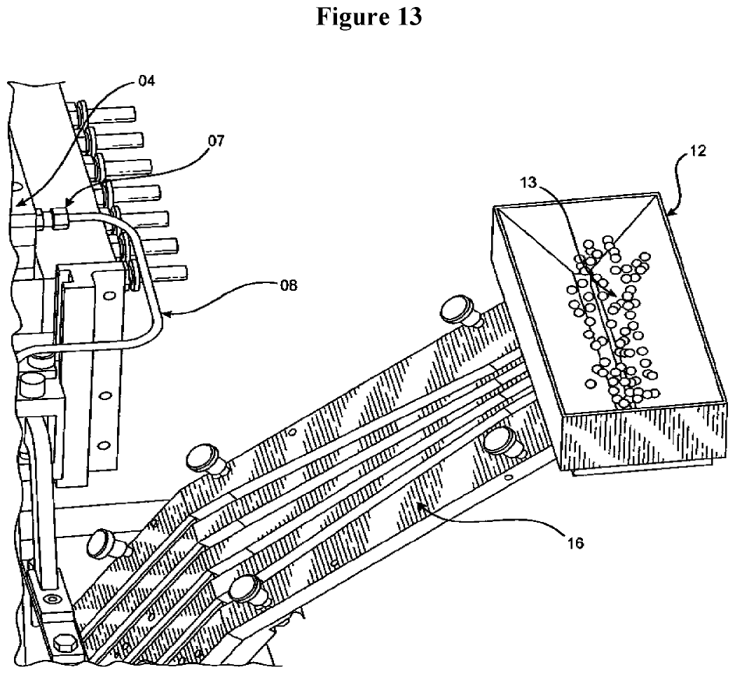

FIG. 13 shows one of the lateral hoppers having smaller solid dosage forms or smaller capsules to be filled inside another softgel capsule. The hopper 12 having capsules 13, are released from the hopper and deposited and guided through guidechannels 16 which in turn leads to the pocket in the rotary mold that is in a tangential position.

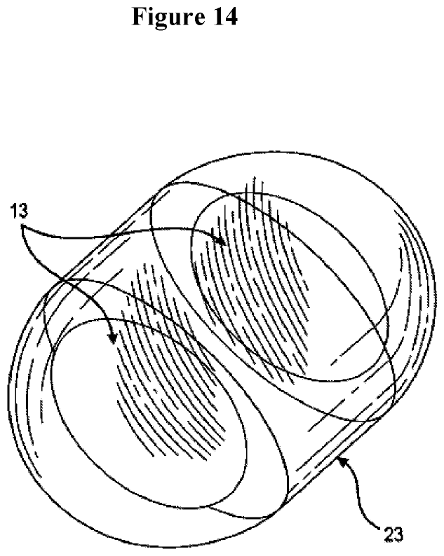

FIG. 14 illustrates a finished capsule of the invention. One or more smaller capsules may be encapsulated in any way into another immersed in a liquid or solution containing a pharmaceutical active ingredient.

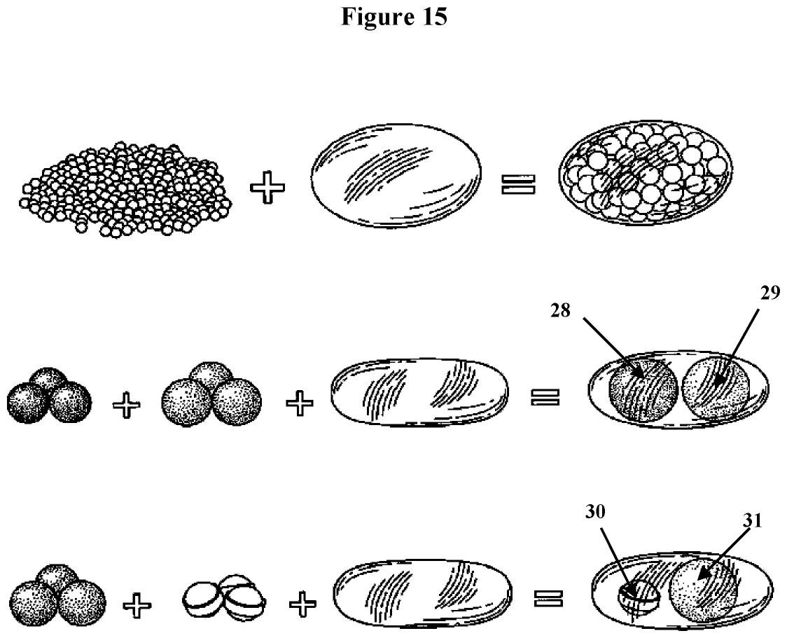

FIG. 15 shows shows several versions of the products of the inversion wherein reference numeral 28 denotes an immediate release solid dosage form and reference numeral 29 denotes an extended release dosage form, while reference numeral 30 and 31 are solid dosage forms of two different drugs exhibiting extended release.

The resulting products of the invention include softgel capsules having incorporated therein another solid dosage form selected from the group consisting: (a) one capsule contains an omega oil and the other solid dosage form is a capsule having a statin; (b) one capsule contains a non-steroidal antiinflammatory and the other solid dosage form contains and antihistamine; and (c) one capsule contains and omega oil and the other solid dosage form contains a salicylate.

Typically the omega oil is an omega-3 oil and the statin is selected from the group consisting of mevastatin, lovastatin, pravastatin, fluvastatin, simvastatin, rosuvastatin, cerivastatin and atorvastatin and derivatives and analogs thereof.

The non-narcotic analgesics/nonsteroidal anti-inflammatory drugs for use in the compositions of the present invention can be selected from the following categories:

(1) the propionic acid derivatives;
(2) the acetic acid derivatives;
(3) the fenamic acid derivatives;
(4) the biphenylcarboxylic acid derivatives; and
(5) the oxicams.

The term “selected NSAID” as used herein is intended to mean any non-narcotic analgesic/non-steroidal anti-inflammatory compound falling within one of the five structural categories but also including aspirin but not acetaminophen and phenacetin.

While some of these compounds are primarily used at the present time as anti-inflammatory agents and others are primarily used as analgesics, in fact all of the contemplated compounds have both analgesic and anti-inflammatory activity and can be used at appropriate dosage levels for either purpose in the compositions and methods of the present invention. The compounds in groups (1) through (4) typically contain a carboxylic acid function; however, those acids are sometimes administered in the form of their pharmaceutically acceptable acid addition or alkali metal salts, e.g., sodium salts.

The propionic acid derivatives for use herein include, but are not limited to, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid. Structurally related propionic acid derivatives having similar analgesic and anti-inflammatory properties are also intended to be encompassed by this group. Presently preferred members of the propionic acid group include ibuprofen, naproxen, flurbiprofen, fenoprofen, ketoprofen and fenbufen.

Thus, “propionic acid derivatives” as defined herein are non-narcotic analgesics/nonsteroidal anti-inflammatory drugs having a free —CH(CH₃)COOH or —CH₂CH₂COOH group (which optionally can be in the form of a pharmaceutically acceptable salt group, e.g. —CH(CH₃)COO⁻H⁺ or —CH₂CH₂COO⁻Na⁺), typically attached directly or via a carbonyl function to a ring system, preferably to an aromatic ring system.

The acetic acid derivatives for use herein include, but are not limited to, indomethacin, sulindac, tolmetin, zomepirac, diclofenac, fenclofenac, alclofenac, ibufenac, isoxepac, furofenac, tiopinac, zidometacin, acemetacin, fentiazac, clidanac and oxpinac. Structurally related acetic acid derivatives having similar analgesic and anti-inflammatory properties are also intended to be encompassed by this group. Presently preferred members of the acetic acid group include tolmetin sodium, zomepirac sodium, sulindac and indomethacin.

Thus, “acetic acid derivatives” as defined herein are non-narcotic analgesics/nonsteroidal anti-inflammatory drugs having a free —CH₂COOH group (which optionally can be in the form of a pharmaceutically acceptable salt group, e.g., —CH₂COO⁻Na⁺), typically attached directly to a ring system, preferably to an aromatic or heteroaromatic ring system.

The fenamic acid derivatives for use herein include, but are not limited to, mefenamic acid, meclofenamic acid, flufenamic acid, niflumic acid and tolfenamic acid. Structurally related fenamic acid derivatives having similar analgesic and anti-inflammatory properties are also intended to be encompassed by this group. Presently preferred members of the fenamic acid group include mefenamic acid and meclofenamate sodium (meclofenamic acid, sodium salt).

Thus, “fenamic acid derivative” as defined herein are non-narcotic analgesics/nonsteroidal anti-inflammatory drugs which contain the basic structure

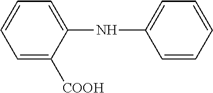 [SMILES: O=C(O)c1ccccc1Nc1ccccc1]

which can bear a variety of substituents and in which the free —COOH group can be in the form of a pharmaceutically acceptable salt group, e.g., —COO⁻Na⁺.

The biphenylcarboxylic acid derivatives for use herein include, but are not limited to, diflunisal and flufenisal. Structurally related biphenylcarboxylic acid derivatives having similar analgesic and anti-inflammatory properties are also intended to be encompassed by this group. Preferred members of this group are diflunisal and flufenisal.

Thus, “biphenylcarboxylic acid derivative” as defined herein are non-narcotic analgesics/nonsteroidal anti-inflammatory drugs which contain the basic structure

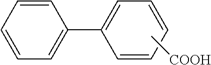

which can bear a variety of substituents and in which the free —COOH group can be in the form of a pharmaceutically acceptable salt group, e.g. —COO⁻Na⁺.

The oxicams for use herein include, but are not limited to, piroxicam, sudoxicam, isoxicam and CP-14, 304. Structurally related oxicams having similar analgesic and anti-inflammatory properties are also intended to be encompassed by this group. A preferred member of this group is piroxicam.

Thus, “oxicams” as defined herein are non-narcotic analgesics/nonsteroidal anti-inflammatory drugs which have the general formula

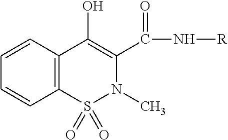

wherein R is an aryl or heteroaryl ring system.

The precise amount of non-narcotic analgesic/non-steroidal anti-inflammatory drug for use in the present compositions will vary depending, for example, on the specific drug chosen, the dosage form thereof, i.e., standard versus sustained release, the condition for which the drug is administered and the size and kind of the mammal.

For humans, typical effective analgesic/anti-inflammatory amounts of presently preferred NSAIDs for use in unit dose compositions of the invention are about 125 to 500 mg diflunisal, about 25 to 100 mg zomepirac sodium, about 50 to 800 mg ibuprofen, most preferably 100-400 mg, about 125 to 500 mg naproxen, about 25 to 50 mg flurbiprofen, and about 50 to 200 mg fenoprofen, about 10 to 20 mg piroxicam, about 125 to 250 mg mefenamic acid, about 100 to 400 mg fenbufen or about 25 to 50 mg ketoprofen; however, greater or lesser amounts can be employed if desired.

The Cox2 inhibitors of the invention are selected from the group consisting of Celecoxib having the formula

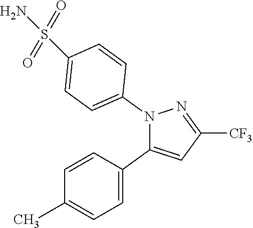 [SMILES: Cc1ccc(-c2cc(C(F)(F)F)nn2-c2ccc(S(N)(=O)=O)cc2)cc1]

Rofecoxib having the formula

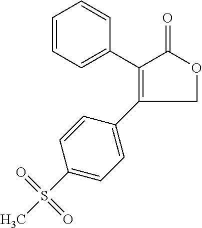 [SMILES: CS(=O)(=O)c1ccc(C2=C(c3ccccc3)C(=O)OC2)cc1]

and valdecoxib having the formula

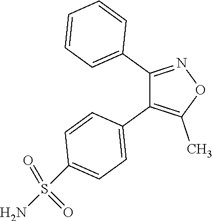 [SMILES: Cc1onc(-c2ccccc2)c1-c1ccc(S(N)(=O)=O)cc1]

Other Cox2 inhibitors also include Parecoxib and MK 663.

The preferred dosage amounts for the Cox2 inhibitors are 100 mg to 200 mg for Celecoxib; 12.5 mg to 25 mg for Rofecoxib and 5-10 mg for Valdecoxib.

The preferred non-steroidal antiinflammatory acid is selected from the group consisting of: ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, indomethacin, sulindac, tolmetin, zomepirac, diclofenac, fenclofenac, alclofenac, ibufenac, isoxepac, furofenac, tiopinac, zidometacin, acemetacin, fentiazac, clidanac, oxpinac, mefenamic acid, meclofenamic acid, flufenamic acid, niflumic acid and tolfenamic acid, diflunisal, flufenisal and piroxicam.

The antihistamine is selected from the group consisting of: diphenhydramine, loratadine, cetirizine, fexofenadine, hydroxyzine, cyproheptadine, chlorphenamine, clemastine and desloratadine.

The salicylate is typically acetylsalicylic acid.

Other active ingredients also include statins selected form the group consisting of mevastatin, lovastatin, pravastatin, fluvastatin, simvastatin, rosuvastatin, cerivastatin and atorvastatin and derivatives and analogs thereof.

The present invention also provides delivery systems which are combined in a highly reliable, easy to use and affordable manufacture that give the resulting dosage form unique characteristics to deliver single or multiple APIs regardless of physical-chemical compatibility and/or stability labilities. The soft-gelatin delivery system can be filled with hydrophilic or lipophilic media to suspend various IR and/or ER dosage forms in drug solutions or plain liquid phases.

The delivery system of the invention is a viable alternative to the manufacturing of IR plus ER combinations in tablets and hard-gelatin capsules while enhancing dosing accuracy and by-passing dissolution barriers and coating issues. It also solves compatibility and stability issues for multivitamins, cold remedies, nutraceuticals and multiple other OTC medications. The invention also allows the formulation of combination products, highly needed to assure patient compliance and allow synergistic clinical effects in a safe and stable dosage form.

The invention also allows for ease of identification by color coding the shell, fill and/or contents minimizing counterfeiting risks.

The drugs in the Table 1 below can be manufactured according to the method of the invention in many different release profiles alone or in combination.

TABLE 1

| | | |
|---|---|---|
| Alendronate | Bupropion HCl | Donepezil HCl |
| Acyclovir | Bupropion HCl | Dorzolamide HCl |
| Acyclovir | Buspirone | Doxazosin Mesylate |
| Albuterol Sulfate | Calcitonin-Salmon | Doxepin |
| Alfuzosin HCl | Calcitriol | Enalapril Maleate |
| Alitretinoin | Calcium Acetate | EnalaprilMaleate- |
| | | Hydrochlorothiazide |
| Allopurinol | Candesartan Cilexetil- | Epinephrine |
| | Hydrochlorothiazide | |
| Alprazolam | Candesartan Cilextil | Eplerenone |
| Altretamine | Captopril | Escitalopram Oxalate |
| Amiodarone | Carbamazepine | Esomeprazole |
| Amitriptyline | Carbidopa/Levo Sr | Estradiol |
| Amlodipine/Valsartan | Carbidopa/Levo | Estropipate |
| Amlodipine Besylate | Carvedilol | Eszopiclone |
| Amlodipine/Valsartan/ | Cetirizine HCl | Etodolac |
| HCTZ | | |
| Amlodipine/Benazepril | Cevimeline HCl | Etodolac |
| Amoxapine | Chlordiazepoxide | Famotidine |
| Anastrazole | Chlorpromazine HCl | Felodipine |
| Antihypertensive | Chlorthalidone | Fenofibrate |
| Combinations | | |
| Aspirin | Cholestyramine | Fenofibric Acid |
| Atenolol | Cilostazol | Ferrous Sulfate |
| Atenolol/Chlorthalidone | Citalopram | Finasteride |
| Atorvastatin Calcium | Clindamycin Phosphate | Flecainide Acetate |
| Augmented | Clonazepam | Fluconazole |
| Betamethasone | | |
| Dipropionate | | |
| Azathioprine | Clonidine HCl | Fluoxetine |
| Azelastine | Clopidogrel Bisulfate | Fluvoxamine Maleate |
| Azelastine Nasal Spray | Colestipol HCl | Folic Acid |
| Baclofen | Decitabine | Furosemide |
| Belladonna Alkaloids | Dexmethylphenidate HCl | Gabapentin |
| With Phenobarbital | | |
| Benazepril HCTZ | Dextroamphetamine Sulfate | Gemfibrozil |
| Benazepril | Dextroamphetamine- | Glimepiride |
| | Amphetamine | |
| Benzonatate | Dextroamphetamine- | Glipizide |
| | Amphetamine | |
| Benzonatate | Diazepam | Glyburide |
| Benztropine | Diclofenac | Glyburide/Metformin |
| Bethanechol | Dicyclomine | Guanfacine |
| Bicalutamide | Dicyclomine | Haloperidol |
| Bisoprolol/Hctz | Digoxin | Hydralazine |
| Brimonidine Tartrate | Diltiazem | Hydrochlorothiazide |
| Bromocriptine | Diltiazem HCl | Hydrocortisone |
| Budesonide | Diphenoxylate/Atropine | Hydroxychloroquine |
| Bupropion HCl | Divalproex | Hydroxyurea |
| Hydroxyzine HCl | Hydroxyzine Pamoate | Ibuprofen |
| Imatinib | Mometasone Furoate | Quinapril HCl |
| Indapamide | Montelukast Sodium | Quinapril/HCTZ |
| Irbesartan | Mycophenolate Mofetil | Rabeprazole Sodium |
| Irbesartan-HCTZ | Nabumetone | Raloxifene |
| Isoniazid | Naproxen | Ramipril |
| Isosorbide Mononitrate | Niacin | Ranolazine |
| Ketotifen Fumarate | Nifedipine | Repaglinide |
| Labetalol HCl | Nilutamide | Risedronate Sodium |
| Lamotrigine | Nitroglycerin | Risperidone |
| Lansoprazole | Norethindrone | Rivastigmine Tartrate |
| Letrozole | Norethindrone/Ethinyl Estradiol | Ropinirole Hydrochloride |
| Levalbuterol HCl | Nortriptyline HCl | Rosuvastatin Calcium |
| Levetiracetam | Nystatin | Sertraline HCl |
| Levocetirizine HCl | Olanzapine | Sildenefil Citrate |
| Levothyroxine | Omega-3 Ethyl Ester | Simvastatin |
| Liothyronine Sodium | Omeprazole | Spironolactone/HCTZ |
| Lisinopril | Ondansetron | Sprintec |
| Lisinopril/HCTZ | Oxandrolone | Sucralfate |
| Lithium Carbonate | Oxybutynin | Sulfamethoxazole/Trimethoprim |
| Loperamide | Pantoprazole Sodium | Sulfasalazine |
| Loratadine | Paroxetine HCl | Sulfasalazine |
| Lorazepam | Pentoxifylline | Sumatriptan Succinate |
| Losartan Potassium | Perphenazine | Tacrolimus |
| Lovastatin | Phenobarbital, Hyoscyamine | Tamoxifen Citrate |
| | Sulfate Atropine Sulfate | |
| | Scopolamine HBr | |
| Loxapine | Phenoxybenzamine | Tamsulosin HCl |
| MagnesiumOxide | Phenytoin Sodium | Telmisartan |
| Meclizine HCl | Pioglitazone HCl | Temazepam |
| Medroxyprogesterone | Potassium Chloride | Terazosin |
| Acetate | | |
| Meloxicam | Potassium Iodide | Testosterone Cypionate |
| Memantine HCl | Pramipexole HCl | Tizanidine |
| Metformin | Pravastatin | Tolterodine Tartrate |
| Metformin | Prazosin | Topiramate |
| Methimazole | Prednisone | Tramadol |
| | | HCl/Acetaminophen |
| Methocarbamol | Primidone | Tramadol |
| Methotrexate | Prochlorperazine | Trandolapril |
| Methylphenidate HCl | Progesterone | Tranylcypromine |
| Metoclopramide HCl | Propafenone HCl | Trazodone |
| Metolazone | Propranolol HCl | Tri-Previfem |
| Metoprolol/HCTZ | Propylthiouracil | Tri-Sprintec |
| Metoprolol Tartrate | Pyridostigmine Bromide | Triamterene |
| Micronized Glyburide | Quetiapine Fumarate | Triamterene/HCTZ |
| Midodrine HCl | Vitamin D3 | Trifluoperazine HCl |
| Minocycline | Vitamin D3 | Trospium Chloride |
| Minocycline HCl | Warfarin Sodium | Valacyclovir HCl |
| Mirtazapine | Zafirlukast | Venlafaxine |
| Valproic Acid | Zaleplon | Verapamil HCl |
| Valsartan | Zidovudine | Vitamin B Complex |
| Hydrochloride | | |
| Valsartan/HCTZ | Zolpidem | Vitamin B-6 |

In additional embodiments of the invention, Applicant has discovered that the softgel capsules of the invention can include several Fixed Dose Combinations FDC (i.e., 2 or more APIs) and biphasic release (1 API in immediate and extended release). The fixed dosage forms can be made in the following release profile modes:

1. Immediate release in the liquid content+extended release in the tablet

2. Extended release in the liquid content+extended release in the tablet

3. Extended release in the liquid content+immediate release in the tablet

The polymer in the capsule shell could be gelatin either porcine or bovine (type A or type B) or a non-animal polymer such as modified starches, carrageenans or alginates.

When tablets are used for incorporation into the capsule product of the invention, they were coated to avoid the migration from the fill content of the capsule to the tablet core and viceversa in order to guarantee the physical and chemical stability of the APIs. The coating that is applied to the tablet consists of two or more polymers in a range of weight gain from (2-10% for each polymer). The dissolution profiles of the resulting tablets are tested according to the USP dissolution conditions corresponding to each monograph. The soft capsule is attached to the stirrer using a device to avoid that the gelatin shell covers the tablet once it gets soft. Dissolution for immediate release, either in the fill content or the coated tablet meets the dissolution criteria of the corresponding USP monograph (Q+/−5% at 30 or 45 min). The dissolution profiles for extended release in the tablet ranges from 6 to 24 hours.

Regarding the embodiments 1, 2 and 3 above, they are further illustrated below with respect to their contents:

1. Softgel capsule includes an immediate release in the liquid content of the capsule and extended release in the tablet that is incorporated within the capsule. The soft gelatin capsule contains an API BCS class I, II, III, IV for both immediate release in the liquid fill content and extended release in the coated tablet. The liquid fill content is an oil, or a polyethylene glycol (PEG) based formulation either as a solution, suspension, emulsion or semisolid. The tablet contains as a matrix for extended release a hydrophilic polymer at different viscosities (1000 to 100.000 cP, preferably Viscosity 2-150.000 mPa·s (2% in water, at 20 C)) and polymer molecular as well as other excipients such as fillers, disintegrants, and lubricants. The tablet is obtained by either of the following processes: wet granulation, dry granulation, spray drying, compression, direct compression, melt granulation or hot melt extrusion. The tablet is coated in order to avoid the migration from the fill content to the tablet core and viceversa in order to guarantee the physical and chemical stability of the APIs. The coating of the tablet consists in two or more polymers in a range of weight gain from (2-10% for each polymer). The dissolution profiles are tested in the USP dissolution conditions corresponding to each monograph. The soft capsule is attached to the stirrer using a device to avoid that the gelatin shell covers the Tablet once it gets soft. Dissolution profiles for extended release in the tablet ranges from 6 to 24 hours.

FIG. 1 shows the extended dissolution profiles of diclofenac tablet cores using a hydrophilic polymer at different viscosities of the polymer for obtaining an extended release matrix. It is appreciated that the dissolution rate can be modified using polymers having different viscosities. In FIG. 2 there is shown an example of the dissolution profile of a dosage form of the invention having a biphasic release from an immediate release fill content and an extended release coated tablet releasing the diclofenac up to 10 hours.

2. Extended release in the liquid content+extended release in the tablet.

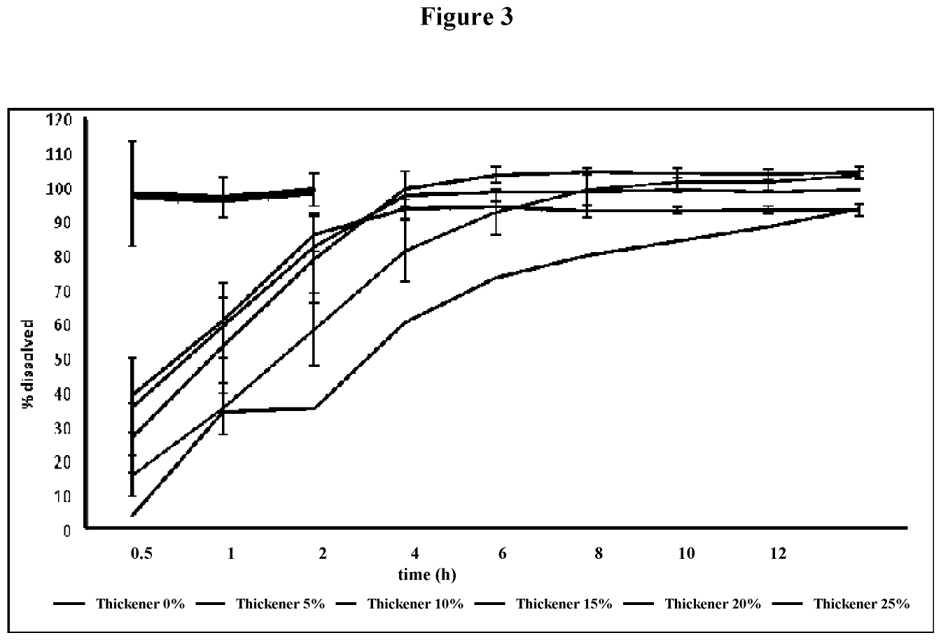

The soft gelatin capsule contains an API BCS class I, II, III, IV for extended release in both the liquid fill content and the coated tablet. The liquid fill content is an oil or a PEG based formulation either as a solution, suspension, emulsion or semisolid. The matrix contains a continuous phase and a thickener at different viscosities and molecular weights. As the thickener concentration increases, the viscosity of the gelled matrix increases and it modifies the dissolution rate of the API through the matrix. FIG. 3 shows the effect of the thickener concentration on the % of dissolved API BCS class II.

The tablet contains as a matrix for extended release a hydrophilic polymer at different viscosities (1000 to 100.000 cP preferably Viscosity 2-150.000 mPa·s (2% in water, at 20 C)) and polymer molecular weights as well as some other excipients such as fillers, disintegrants, and lubricants. The tablet is obtained by either of the following processes: wet granulation, dry granulation, spry drying, compression, direct compression, melt granulation or hot melt extrusion.

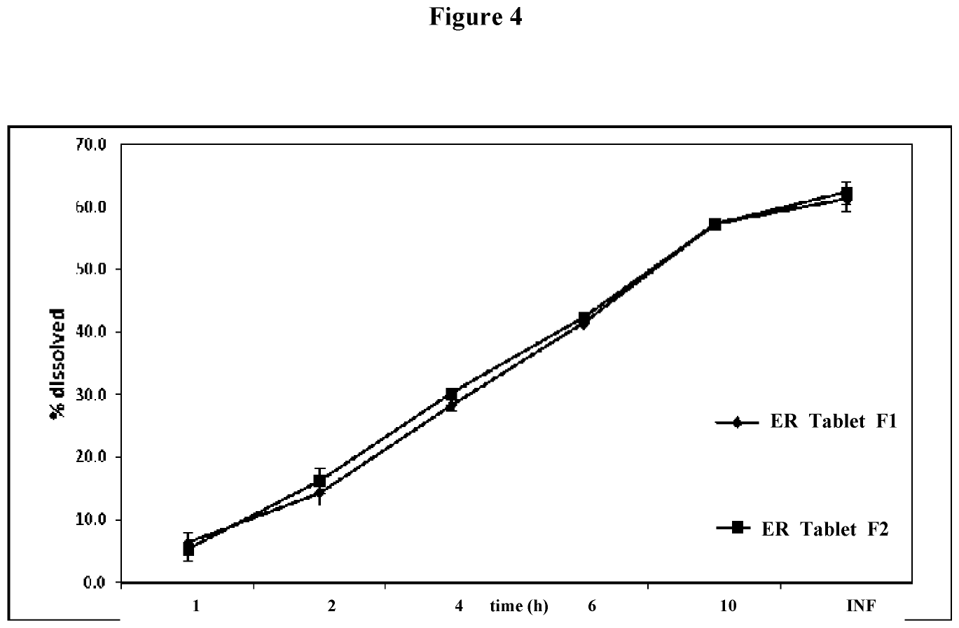

FIG. 4 shows the dissolution profiles of the coated extended release tablets of two core formulations F1 and F2 up to 10 hours. Error bars show an acceptable variability. Dose of the Tablet is 63% of the total dose in the product of the invention.

3. Extended release in the liquid content+immediate release of the tablet.

The dissolution profiles for extended release fill contents are shown in FIG. 3 for an API BCS class II up to 12 hours. Dissolution profiles for immediate release tablets have been shown previously (atorvastatina 20 mg en Lipomega) and meet the dissolution criteria of the corresponding USP monograph (Q+/−5% at 30 or 45 min).

The drug carrier solvent system in the softgel capsule of the invention may include 5% to 45% by weight glycerin, more preferably about 10% to 40% by weight of glycerin and most preferably 15% to 35% or other similar solvent such as propylene glycol or other low molecular weight polyethylene glycols. The polyethylene glycols useful herein are those which are liquids at room temperature or have a melting point slightly thereabove. Preferred are the polyethylene glycols having a molecular weight range from about 300 to about 1000 and corresponding n values from about 6 to about 20. More preferred are the polyethylene glycols having a molecular weight range from about 400 to about 1000 and corresponding n values from about 8 to about 20. Most preferred are the polyethylene glycols having a molecular weight range from about 600 to about 1000 and corresponding n values from about 12 to about 20. Most especially preferred is a polyethylene glycol having a molecular weight of about 600 and a corresponding n value of about 12. Liquid and low-melting polyethylene glycols are commercially available from Union Carbide (Danbury, Conn.) under the Carbowax™. See Carbowax™ Polyethylene Glycols.

The solvent system further includes 5%-50% by weight water, more preferably 10%-45% by weight water and most preferably 15% to 35% by weight water.

In the cases wherein the NSAID's have a carboxyl or an acidic function, the solvent system of the invention also includes about 0.05-1.0 mole of hydroxide ions for each molar equivalent of the acidic medicine. Hydroxide ions originated, for example, from sodium and/or potassium hydroxide, are used together with water. The most preferred alkaline hydroxide is potassium hydroxide.

The solvent system of the invention may also include optionally 0.5%-25% by weight of polyvinylpyrrolidone (PVP). The soluble forms of polyvinylpyrrolidone are preferred for use in the present invention. Preferred are soluble polyvinylpyrrolidones having an average molecular weight in the range from about 3000 to about 1,000,000; more preferred are those having an average molecular weight in the range from about 7500 to about 50,000; and most preferred are those having an average molecular weight of about 30,000. Moreover, mixtures of two or more soluble polyvinylpyrrolidones of different average molecular weight can be employed.

Other components which can be incorporated into the compositions of the instant invention include colorings, flavorings, preservatives, lubricants, flow-enhancers, filling aids, antioxidants, essences, and other aesthetically pleasing components.

The solubilized pharmaceutical compositions of the present invention can be encapsulated within any conventional soft gelatin shell that is capable of substantially containing the composition for a reasonable period of time. The soft gelatin shells of the instant invention can be prepared by combining appropriate amounts of gelatin, water, plasticizer, and any optional components in a suitable vessel and agitating and/or stirring while heating to about 65° C. until a uniform solution is obtained. This soft gelatin shell preparation can then be used for encapsulating the desired quantity of the solubilized fill composition employing the methodology to make soft gelatin capsules having incorporated within an additional smaller solid dosage form.

The fill formulation containing one of the actives is encapsulated into one-piece gelatin sheath or shell that includes a plasticizer to control the softness and flexibility of the sheath, water, and optionally, other additives, such as flavorants, colorants, opacifiers, etc. The softgel capsules having incorporated smaller dosage forms are produced as described above using the apparatus of the invention.

Suitable sheath formulations may include from about 30 to about 50% by weight gelatin; at least 18% by weight, and preferably up to about 40% by weight, of a plasticizer; and from about 20 to about 50% by weight water. These formulations, when formed into capsules and dried, will result in capsule sheaths comprised of from about 40 to about 75% by weight gelatin; from about 18% to about 40% by weight plasticizer; and from about 5 to about 15% by weight water.

The gelatin will normally have a bloom in the range of from about 140 to about 280, and may be Type A or B gelatins or a mixture thereof. Limed bone, acid bone, fish and/or pig skin gelatins may be used.

The gelatin capsules containing the smaller solid dosage forms are formed into the desired shape and size so that they can be readily swallowed. The soft gelatin capsules of the instant invention are of a suitable size for easy swallowing and typically contain from about 100 mg to about 2000 mg of the solubilized pharmaceutical active composition and 20 mg to about 1000 mg of a smaller capsule or tablet having immediate release or extended release profile. The resulting soft gelatin capsule is soluble in water and in gastrointestinal fluids. Upon swallowing the capsule, the gelatin shell rapidly dissolves or ruptures in the gastrointestinal tract thereby introducing the pharmaceutical actives into the physiological system.

The solid dosage forms of the invention are coated with film formers to provide extended release properties. The film-forming materials of the invention comprise at least one component selected from the group consisting of gelatin, starch, carrageenans, gums or synthetic materials such as hydroxypropyl-methylcellulose (HPMC), other hydroxyalkylated celluloses and the like. The film-forming material typically has an aqueous base and is considered to be ingestible. As used herein, the term “ingestible” is used to indicate a film-forming material that dissolves under conditions simulating the human digestion tract or water.

The extended release controlling polymer is selected from the group consisting of hydroxypropyl cellulose (HPC), hydroxypropylmethyl cellulose (HPMC), ethyl cellulose (EC), cellulose acetate, acrylic polymers, polyvinylpyrrolidone (PVP), or combinations thereof. Currently preferred release controlling polymers are hydroxypropyl cellulose (HPC), hydroxypropylmethyl cellulose (HPMC), ethyl cellulose (EC), and combinations thereof. Each possibility represents a separate embodiment of the present invention.

In some embodiments, the smaller dosage form is in a form selected from extended release (ER) beads, mini-tablets, double-layer tablets, hard or soft gelatin capsule, a pellet, or combinations thereof. In some embodiments, the composition of the invention is in the form of ER beads or mini tablets filled into hard or soft gelatin capsules or compressed into dispersible tablets. Mixtures of any of the above are also contemplated. Each possibility represents a separate embodiment of the invention.

The following examples further describe and demonstrate embodiments within the scope of the present invention. The examples are given solely for the purpose of illustration and are not to be construed as limitations of the present invention, as many variations thereof are possible without departing from the spirit and scope of the invention.

### EXAMPLES

The following procedure is used throughout the examples below to dissolve the active principle in the solvent system which is then encapsulated in the softgel.

Mix the PEG and the Glycerin under moderate agitation, heat to a temperature ranging from 55° C.+/−5° C. Add the active principle and strongly mix to have a good dispersion. The Potassium Hydroxide was slowly added in an aqueous solution; the mixture is then strongly agitated until a clear transparent solution is obtained. Stop the heating and keep agitating the solution until it is at room temperature. The active material solution is suitable to be encapsulated in soft gelatin capsules.

### Example 1

| COMPONENTS | AMOUNT/mg | |
|---|---|---|
| Ibuprofen | 200.0 | mg |
| Potassium Hydroxide | 24.0 | mg |
| Water | 18.0 | mg |
| Glycerin | 17.0 | mg |
| PEG | 59.0 | mg |
| Total | 318.0 | mg |

### Example 2

| COMPONENTS | AMOUNT/mg | |
|---|---|---|
| Ibuprofen | 200.0 | mg |
| Potassium Hydroxide | 21.0 | mg |
| Water | 23.6 | mg |
| Glycerin | 23.0 | mg |
| PEG | 50.4 | mg |
| Total | 318.0 | mg |

### Example 3

| COMPONENTS | AMOUNT/mg | |
|---|---|---|
| Naproxen | 200.0 | mg |
| Potassium Hydroxide | 40.0 | mg |
| Water | 18.0 | mg |
| Glycerin | 17.0 | mg |
| PEG | 59.0 | mg |
| Total | 334.0 | mg |

### Example 4

| COMPONENTS | AMOUNT/mg | |
|---|---|---|
| Celecoxib | 200.0 | mg |
| Water | 18.0 | mg |
| Glycerin | 17.0 | mg |
| PEG | 59.0 | mg |
| Total | 294.0 | mg |

### Example 5

| COMPONENTS | AMOUNT/mg | |
|---|---|---|
| Rofecoxib | 25.0 | mg |
| Water | 18.0 | mg |
| Glycerin | 17.0 | mg |
| PEG | 59.0 | mg |
| Total | 119.0 | mg |

### Example 6

| COMPONENTS | AMOUNT/mg | |
|---|---|---|
| Ibuprofen | 200.0 | mg |
| Potassium Hydroxide | 24.0 | mg |
| Water | 18.0 | mg |
| Glycerin | 17.0 | mg |
| PVP Avg. MW 30,000 | 20 | mg |
| PEG | 59.0 | mg |
| Total | 338.0 | mg |

### Example 7

### Soft Gelatin Capsule Containing a Solubilized Ibuprofen Composition

A soft gelatin mixture is first prepared from the following ingredients.

| INGREDIENT | WEIGHT % |
|---|---|
| Gelatin | 48.00 |
| Glycerin | 14.00 |
| Water | QS 100 |

the above ingredients are combined in a suitable vessel and heated with mixing at about 65° C. to form a uniform solution. Using standard encapsulation methodology, the resulting solution is used to prepare soft gelatin capsules containing approximately 318 mg of the composition as prepared in Example 1. The resulting soft gelatin ibuprofen capsules are suitable for oral administration.

Tabs are made with the formulations of Examples 8 to Examples 13 and incorporated into a softgel capsule containing an immediate release formulation of diclofenac in PEG.

### Example 8

| INGREDIENTS | Mg/Tab | % |
|---|---|---|
| Sodium Diclofenac | 75.0 | 31.3 |
| Lactose monohydrate | 87.0 | 36.3 |
| Hydroxypropyl methylcellulose | 2.4 | 1 |
| Viscosity (mPa's) = 4-6 | | |
| Hydroxypropyl methylcellulose | 72.0 | 30 |
| Viscosity (mPa's) = 80-120 | | |
| Talc | 2.4 | 1 |
| Magnesium Stearate | 1.2 | 0.5 |
| Total | 240 | 100 |

### Example 9

| INGREDIENTS | Mg/Tab | % |
|---|---|---|
| Sodium Diclofenac | 75.0 | 31.3 |
| Lactose monohydrate | 87.0 | 36.3 |
| Hydroxypropyl methylcellulose | 2.4 | 1 |
| Viscosity (mPa's) = 4-6 | | |
| Hydroxypropyl methylcellulose | 72.0 | 30 |
| Viscosity (mPa's) = 2663-4970 | | |
| Talc | 2.4 | 1 |
| Magnesium Stearate | 1.2 | 0.5 |
| Total | 240 | 100 |

### Example 10

| INGREDIENTS | Mg/Tab | % |
|---|---|---|
| Sodium Diclofenac | 75.0 | 31.3 |
| Lactose monohydrate | 87.0 | 36.3 |
| Hydroxypropyl methylcellulose | 2.4 | 1 |
| Viscosity (mPa's) = 4-6 | | |
| Hydroxypropyl methylcellulose | 72.0 | 30 |
| Viscosity (mPa's) = 72,750-135,800 | | |
| Talc | 2.4 | 1 |
| Magnesium Stearate | 1.2 | 0.5 |
| Total | 240 | 100 |

### Example 11

| INGREDIENTS | Mg/Tab | % |
|---|---|---|
| Sodium Diclofenac | 75.0 | 31.3 |
| Pregelatinized Starch | 87.0 | 36.3 |
| Hydroxypropyl methylcellulose | 2.4 | 1 |
| Viscosity (mPa's) = 4-6 | | |
| Hydroxypropyl methylcellulose | 72.0 | 30 |
| Viscosity (mPa's) = 80-120 | | |
| Talc | 2.4 | 1 |
| Magnesium Stearate | 1.2 | 0.5 |
| Total | 240 | 100 |

### Example 12

| INGREDIENTS | Mg/Tab | % |
|---|---|---|
| Sodium Diclofenac | 75.0 | 31.3 |
| Pregelatinized Starch | 87.0 | 36.3 |
| hydroxypropyl methylcellulose | 2.4 | 1 |
| Viscosity (mPa's) = 4-6 | | |
| Hydroxypropyl methylcellulose | 72.0 | 30 |
| Viscosity (mPa's) = 2663-4970 | | |
| Talc | 2.4 | 1 |
| Magnesium Stearate | 1.2 | 0.5 |
| Total | 240 | 100 |

### Example 13

| INGREDIENTS | Mg/Tab | % |
|---|---|---|
| Sodium Diclofenac | 75.0 | 31.3 |
| Pregelatinized Starch | 87.0 | 36.3 |
| hydroxypropyl methylcellulose | 2.4 | 1 |
| Viscosity (mPa's) = 4-6 | | |
| hydroxypropyl methylcellulose | 72.0 | 30 |
| Viscosity (mPa's) = 72,750-135,800 | | |
| Talc | 2.4 | 1 |
| Magnesium Stearate | 1.2 | 0.5 |
| Total | 240 | 100 |

Additional Examples of the invention are summarized in the table 2 below.

TABLE 2

| PRODUCTS OF THE INVENTION | PROFILE CHARACTERISTICS |
|---|---|
| Fenofibric Acid + Rosuvastatin | Different Release Modes |
| APAP - Acetaminophen (500 mg) | The product looks like a soft capsule |
| APAP - PM Acetaminophen (500 mg) + | Different Release Modes |
| Diphenhydramine (25 mg) | |
| Atorvastatin (40 mg) + Omega 55% (600 mg) | At Least one of the APIs is a liquid |
| Atorvastatin (20 mg) + Aspirin (100 mg) + Omega | At Least one of the APIs is a liquid |
| 84% | |
| Atorvastatin (40 mg) + Aspirin (100 mg) + Omega | At Least one of the APIs is a liquid |
| 84% | |
| Butilhioscina (20 mg) + Naproxen Sódico (275 mg) | Incompatible APIs |
| Cetirizine (5 mg) + Phenylephrine (15 mg) | Different Release Modes |
| Docusate sodium (100 mg) + Simethicone (125 mg) | The product looks like a soft capsule |
| Dutasteride + Tamsulosine | The product looks like a soft capsule |
| Esomeprazole (20 mg) | The product looks like a soft capsule |
| Esomeprazole (40 mg) + magnesium hydroxide (211 mg) | The product looks like a soft capsule |
| Esomeprazole Magnesium + Naproxen | Incompatible APIs |
| Ibuprofen XR | Different Release Modes |
| Ibuprofen (400 mg) o (200 mg) + Hisocine | Incompatible APIs |
| (20 mg) o (10 mg) | |
| Ibuprofen + Hisocine + Acetaminophen | Incompatible APIs |
| Ibuprofen (400 mg) + Hisocine (20 mg) + | Incompatible APIs |
| Caffeine (250 mg) (2 tablets 125 mg) | |
| Ibuprofen (400 mg) + Acetaminophen (250 mg) | The product looks like a soft capsule |
| (2 tablets 125 mg) | |
| Ibuprofen (200 mg) + Metocarbamol (500 mg) | Incompatible APIs |
| Ibuprofen + Dextrometorphan + Levocetirizine + | The product looks like a soft capsule |
| Phenylephrine - Modified Release | |
| Omeprazole + Aluminio o Magnesio | The product looks like a soft capsule |
| Omeprazol + Aluminum ro Magnesium hydroxide + | The product looks like a soft capsule |
| Simethicone | |
| Rifaximine XR | Different Release Modes |
| Simethicone + Probiotics | Incompatible APIs |
| Trimebutine + Probiotics | Incompatible APIs |
| Trimebutine + Simethicona + Probiotics | Incompatible APIs |
| Valsartan + Hydrochlorothiazide | Incompatible APIs |
| Valsartan + Amlodipine | Incompatible APIs |
| Valsartan + Amlodipine + Hydroclorothiazide | Incompatible APIs |
| Valsartan + Amlodipine + Hydroclorotiazide + ASA | Incompatible APIs |
| Zolpidem (10 mg) | Different Release Modes |

It should be noted that the softgel capsule of the invention contains one or more solid dosage forms.

All literature and similar materials cited in this application including, but not limited to, patents, patent applications, articles, books, treatises, and internet web pages, regardless of the format of such literature and similar materials, are expressly incorporated by reference in their entirety for any purpose as if they were entirely denoted. In the event that one or more of the incorporated literature and similar materials defines or uses a term in such a way that it contradicts that term's definition in this application, this application controls.

Although the foregoing description contains many specifics, these should not be construed as limiting the scope of the present invention, but merely as providing illustrations of some of the presently preferred embodiments. Similarly, other embodiments may be devised without departing from the spirit or scope of the present invention. Features from different embodiments may be employed in combination. The scope of the invention is, therefore, indicated and limited only by the appended claims and their legal equivalents rather than by the foregoing description. All additions, deletions and modifications to the invention as disclosed herein which fall within the meaning and scope of the claims are to be embraced thereby.

What is being claimed is:

1. A softgel capsule having incorporated within said capsule: (i) an active pharmaceutical ingredient in a liquid carrier exhibiting release profiles selected from the group consisting of immediate release, extended release and combinations thereof; and (ii) one or more smaller capsules or smaller solid dosage forms wherein said smaller capsules or smaller solid dosage forms have active ingredients that are compatible or not compatible with each other or with another active within the softgel capsule and wherein said smaller capsules or smaller solid dosage forms exhibit release profiles selected from the group consisting of extended release, and immediate release and combinations thereof.

2. The softgel capsule of claim 1, wherein the liquid carrier is an omega-3 oil,

3. The softgel capsule of claim 1, wherein the liquid carrier is polyethylene glycol.

4. The softgel capsule of claim 1, wherein the active pharmaceutical ingredient (i) is a non-steroidal antiinflammatory.

5. The softgel capsule of claim 4, wherein the non-steroidal antiinflammatory is selected from the group consisting of ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, indomethacin, sulindac, tolmetin, zomepirac, diclofenac, fenclofenac, alclofenac, ibufenac, isoxepac, furofenac, tiopinac, zidometacin, acemetacin, fentiazac, clidanac, oxpinac, mefenamic acid, meclofenamic acid, flufenamic acid, niflumic acid and tolfenamic acid, diflunisal, flufenisal and piroxicam.

6. The softgel capsule of claim 1, wherein the active pharmaceutical ingredient (i) is an antihistamine.

7. The softgel capsule of claim 6, wherein said anti-histamine is selected from the group consisting of diphenhydramine, loratadine, cetirizine, fexofenadine, hydroxyzine, cyproheptadine, chlorphenamine, clemastine and desloratadine.

8. The softgel capsule of claim 1, wherein the active pharmaceutical ingredient (i) is s statin.

9. The softgel capsule of claim 8, wherein said statin is selected from the group consisting of mevastatin, lovastatin, pravastatin, fluvastatin, simvastatin, rosuvastatin, cerivastatin and atorvastatin and derivatives and analogs thereof

10. A softgel capsule having incorporated within said capsule: (i) an immediate release active pharmaceutical ingredient in a liquid carrier; and (ii) one or more smaller capsules or smaller solid dosage forms wherein said smaller capsules or smaller solid dosage forms have active ingredients that are compatible or not compatible with each other or with another active within the softgel capsule and wherein said smaller capsules or smaller solid dosage forms exhibit release profiles selected from the group consisting of extended release, and immediate release and combinations thereof

11. The softgel capsule of claim 10, wherein the liquid carrier is an omega-3 oil,

12. The softgel capsule of claim 10, wherein the liquid carrier is polyethylene glycol.

13. The softgel capsule of claim 10, wherein the active pharmaceutical ingredient (i) is a non-steroidal antiinflammatory.

14. The softgel capsule of claim 13, wherein the non-steroidal antiinflammatory is selected from the group consisting of ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, indomethacin, sulindac, tolmetin, zomepirac, diclofenac, fenclofenac, alclofenac, ibufenac, isoxepac, furofenac, tiopinac, zidometacin, acemetacin, fentiazac, clidanac, oxpinac, mefenamic acid, meclofenamic acid, flufenamic acid, niflumic acid and tolfenamic acid, diflunisal, flufenisal and piroxicam.

15. The softgel capsule of claim 10, wherein the active pharmaceutical ingredient (i) is an antihistamine.

16. The softgel capsule of claim 15, wherein said anti-histamine is selected from the group consisting of diphenhydramine, loratadine, cetirizine, fexofenadine, hydroxyzine, cyproheptadine, chlorphenamine, clemastine and desloratadine.

17. The softgel capsule of claim 10, wherein the active pharmaceutical ingredient (i) is s statin.

18. The softgel capsule of claim 17, wherein said statin is selected from the group consisting of mevastatin, lovastatin, pravastatin, fluvastatin, simvastatin, rosuvastatin, cerivastatin and atorvastatin and derivatives and analogs thereof

19. The softgel capsule of claim 10, wherein said smaller solid dosage form consists of an extended release diclofenac tablet.

20. The softgel capsule of claim 10, wherein said smaller solid dosage form consists of an extended release statin.
